(19) United States
(12) Patent Application Publication
(10) Pub. No.: US 2025/0041618 A1
(43) Pub. Date: Feb. 6, 2025

(54) PACKAGE, PLASMA IRRADIATION MODULE, AND PLASMA IRRADIATION SYSTEM

(71) Applicant: NITERRA CO., LTD., Nagoya-shi, Aichi (JP)

(72) Inventors: Yoshihiro NAKANO, Nagoya-shi, Aichi (JP); Yuji MATSUMOTO, Nagoya-shi, Aichi (JP); Kei TAKAHASHI, Nagoya-shi, Aichi (JP)

(73) Assignee: NITERRA CO., LTD., Nagoya-shi, Aichi (JP)

(21) Appl. No.: 18/710,531

(22) PCT Filed: Dec. 2, 2022
(86) PCT No.: PCT/JP2022/044534

§ 371 Date: May 15, 2024

(30) Foreign Application Priority Data
Oct. 17, 2022 (JP) 2022-166210

(51) Int. Cl. A61N 1/44; A61N 1/08

(52) U.S. Cl. CPC A61N 1/44; A61N 1/08

(57) ABSTRACT

A package used for a plasma irradiation system includes a plasma irradiation device which includes a base portion functioning as a grip portion, and a detachable component which is detachably attached to the base portion and has a discharging opening; and a control apparatus to which the plasma irradiation device is connected and which controls the plasma irradiation device. The package includes the detachable component, a packaging material, and an information section containing a piece of information which is readable by the control apparatus. The information section is provided at least on the detachable component, on the packaging material, or in the packaging material and includes at least one of a memory section for memorizing the number of times of use of the detachable component, and an identification section in which a piece of identification information of the detachable component has been recorded.

### TECHNICAL FIELD

The present invention relates to a package, a plasma irradiation module, and a plasma irradiation system.

### BACKGROUND ART

Patent Literature 1 discloses a plasma irradiation device which discharges plasma. The plasma irradiation device is used in operations such as dental treatment. Scatters from an object irradiated with plasma are likely to adhere to a periphery of a portion of the plasma irradiation device from which plasma is discharged. In the plasma irradiation device disclosed in Patent Literature 1, a nozzle including the portion from which plasma is discharged is configured to be detachable. Therefore, when scatters from the object irradiated with plasma have adhered to the nozzle, replacement of only the nozzle is possible.

### CITATION LIST

### Patent Literature

Patent Literature 1: JP2020-10770A

### SUMMARY OF INVENTION

### Technical Problem

In the case where a plasma irradiation device has a structure in which a detachable component is detachably attached to a forward end as shown in Patent Literature 1, it is desired to easily manage the number of times the detachable component has been used.

An object of the present invention is to provide a technique for facilitating management of the number of times of use of a detachable component provided at a forward end of a plasma irradiation device.

### SUMMARY OF INVENTION

A package of the present invention is a package used for a plasma irradiation system,
wherein the plasma irradiation system comprises:
a plasma irradiation device which includes a base portion functioning as a grip portion, and a detachable component which is detachably attached to the base portion and has a discharging opening for discharging plasma; and
a control apparatus to which the plasma irradiation device is connected and which controls the plasma irradiation device,
wherein the package comprises:
the detachable component:
a packaging material for packaging the detachable component; and
an information section containing a piece of information which can be read by the control apparatus, and
wherein the information section is provided at least on the detachable component, on the packaging material, or in the packaging material and includes at least one of a memory section for memorizing the number of times of use of the detachable component, and an identification section in which a piece of identification information for identifying the detachable component has been recorded.

A plasma irradiation module of the present invention comprises:
the package; and
a base portion constituting body which constitutes the base portion.

A plasma irradiation system of the present invention comprises:
the plasma irradiation module; and
the control apparatus,
wherein the information section includes the memory section, and
wherein the control apparatus performs a reading processing of reading the number of times of use of the detachable component memorized in the memory section, a memorizing processing of updating the number of times of use of the detachable component memorized in the memory section, and a permission processing of permitting operation of the plasma irradiation device on the basis of the number of times of use of the detachable component memorized in the memory section.

A plasma irradiation system of the present invention comprises:
the plasma irradiation module; and
the control apparatus,
wherein the information section includes the identification section, and
wherein the control apparatus performs a reading processing of reading the piece of identification information for identifying the detachable component recorded in the identification section, a memorizing processing of memorizing the number of times of use of the detachable component in an apparatus-side memory section in such a manner that the number of times of use is made to correspond to the read identification information, and a permission processing of permitting operation of the plasma irradiation device on the basis of the number of times of use of the detachable component memorized in the apparatus-side memory section.

### Advantageous Effect of Invention

The present invention can facilitate management of the number of times of use of the detachable component provided at the forward end of the plasma irradiation device.

### BRIEF DESCRIPTION OF DRAWINGS

FIG. 1 is a diagram schematically illustrating a plasma irradiation system of a first embodiment.

FIG. 2 is a side view illustrating a state before attachment of a detachable component to a base portion.

FIG. 3 is a side sectional view illustrating a state in which the detachable component has been attached to the base portion.

FIG. 4 is a side sectional view of the detachable component.

FIG. 5 is a block diagram illustrating the electrical configuration of the plasma irradiation system of the first embodiment.

FIG. 6 is a flowchart illustrating the flow of processing performed by a control apparatus.

FIG. 7 is a diagram schematically illustrating a plasma irradiation system of a second embodiment.

FIG. 8 is a block diagram illustrating the electrical configuration of the plasma irradiation system of the second embodiment.

FIG. 9 is a diagram schematically illustrating a plasma irradiation system of a third embodiment.

FIG. 10 is a block diagram illustrating the electrical configuration of the plasma irradiation system of the third embodiment.

FIG. 11 is an explanatory view schematically illustrating a package of a fourth embodiment.

FIG. 12 is a side sectional view of a detachable component of a fifth embodiment.

FIG. 13 is a side sectional view of a detachable component of a sixth embodiment.

### DESCRIPTION OF EMBODIMENTS

In the following description, embodiments of the present disclosure are listed and illustrated.

[1] A package used for a plasma irradiation system,
wherein the plasma irradiation system comprises:
a plasma irradiation device which includes a base portion functioning as a grip portion, and a detachable component which is detachably attached to the base portion and has a discharging opening for discharging plasma; and
a control apparatus to which the plasma irradiation device is connected and which controls the plasma irradiation device,
wherein the package comprises:
the detachable component;
a packaging material for packaging the detachable component; and
an information section containing a piece of information which can be read by the control apparatus, and
wherein the information section is provided at least on the detachable component, on the packaging material, or in the packaging material and includes at least one of a memory section for memorizing the number of times of use of the detachable component, and an identification section in which a piece of identification information for identifying the detachable component has been recorded.

The information section is provided on the detachable component, on the packaging material with which the detachable component is packaged, or in the packaging material with which the detachable component is packaged, so that the information section is made to correspond to the detachable component. Namely, in the configuration in which the information section includes the memory section, the number of times of use of the detachable component can be managed by using the memory section made to correspond to the detachable component. Also, in the configuration in which the information section includes the identification section, it is possible to specify the piece of identification information for identifying the detachable component on the control apparatus side by using the identification section made to correspond to the detachable component, and to manage the number of times of use of the detachable component for each piece of identification information. Thus, the above-described package can facilitate management of the number of times of use of the detachable component.

[2] The package described in [1], wherein the plasma irradiation device has an electric discharge section for generating plasma, and the electric discharge section is provided in the detachable component.

By virtue of this configuration, since the electric discharge section is disposed near the discharging opening, plasma is more likely to be generated in a region near the discharging opening.

[3] The package described in [2], wherein the information section includes a memory circuit for memorizing a piece of information and is provided at least on the packaging material or in the packaging material.

The information memorized in the memory circuit of the information section is likely to receive an influence of electromagnetic noise produced stemming from the voltage applied to the electric discharge section. Since the above-described package is configured such that the information section is provided at least on the packaging material or in the packaging material, the information section can be separated from the detachable component in which the electric discharge section is provided. Therefore, the above-described package can reduce the influence on the information in the information section of the electromagnetic noise produced stemming from the voltage applied to the electric discharge section.

[4] A plasma irradiation module which comprises the package described in any of [1] to [3] and a base portion constituting body which constitutes the base portion.

[5] The plasma irradiation module described in [4], further comprising a second information section which is provided in the base portion constituting body and contains a piece of information which can be read by the control apparatus,

wherein the second information section includes at least one of a second memory section for memorizing the number of times of use of the base portion constituting body, and a second identification section in which a piece of identification information for identifying the base portion constituting body has been recorded.

In the configuration in which the second information section includes the second memory section, the above-described plasma irradiation module makes it possible to manage the number of times of use of the base portion constituting body by using the second memory section. Also, in the configuration in which the second information section includes the second identification section, the above-described plasma irradiation module makes it possible to specify the identification information recorded in the second identification section on the control apparatus side and to manage the number of times of use of the base portion constituting body for each piece of identification information. Thus, the above-described plasma irradiation module facilitates management of not only the number of times of use of the detachable component but also the number of times of use of the base portion constituting body.

[6] A plasma irradiation system comprising:
the plasma irradiation module described in [4] or [5]; and
the control apparatus,
wherein the information section includes the memory section, and
wherein the control apparatus performs a reading processing of reading the number of times of use of the detachable component memorized in the memory section, a memorizing processing of updating the number of times of use of the detachable component memorized in the memory section, and a permission processing of permitting operation of the plasma irradiation device on the basis of the number of times of use of the detachable component memorized in the memory section.

Since the above-described plasma irradiation system permits operation of the plasma irradiation device on the basis of the number of times of use of the detachable component, upon arrival of a time at which operation of the plasma irradiation device is not permitted, a new information section becomes necessary. For preparation of a new information section, a new package must be opened. Opening of the new package results in replacement of the detachable component. Namely, the above-described plasma irradiation system can prompt a user to replace the detachable component on the basis of the number of times of use of the detachable component.

[7] The plasma irradiation system described in [6], further comprising a second information section which is provided in the base portion constituting body and contains a piece of information which can be read by the control apparatus,
wherein the second information section includes a second memory section for memorizing the number of times of use of the base portion constituting body, and
wherein, in the reading processing, the control apparatus reads the number of times of use of the detachable component memorized in the memory section and the number of times of use of the base portion constituting body memorized in the second memory section,
in the memorizing processing, the control apparatus updates the number of times of use of the detachable component memorized in the memory section and the number of times of use of the base portion constituting body memorized in the second memory section, and
in the permission processing, the control apparatus permits operation of the plasma irradiation device on the basis of the number of times of use of the detachable component memorized in the memory section and the number of times of use of the base portion constituting body memorized in the second memory section.

The above-described plasma irradiation system requires replacement of the detachable component when the number of times of use of the detachable component reaches an upper limit and requires replacement of the base portion constituting body when the number of times of use of the base portion constituting body reaches an upper limit. Therefore, the above-described plasma irradiation system can prompt the user to replace the detachable component on the basis of the number of times of use of the detachable component and can also prompt the user to replace the base portion constituting body on the basis of the number of times of use of the base portion constituting body.

[8] A plasma irradiation system comprising:
the plasma irradiation module described in [4] or [5]; and
the control apparatus,
wherein the information section includes the identification section, and
wherein the control apparatus performs a reading processing of reading the piece of identification information for identifying the detachable component recorded in the identification section, a memorizing processing of memorizing the number of times of use of the detachable component in an apparatus-side memory section in such a manner that the number of times of use is made to correspond to the read identification information, and a permission processing of permitting operation of the plasma irradiation device on the basis of the number of times of use of the detachable component memorized in the apparatus-side memory section.

Since the above-described plasma irradiation system permits operation of the plasma irradiation device on the basis of the number of times of use of the detachable component, upon arrival of a time at which operation of the plasma irradiation device is not permitted, a new information section becomes necessary. For preparation of a new information section, a new package must be opened. Opening of the new package results in replacement of the detachable component. Namely, the above-described plasma irradiation system can prompt a user to replace the detachable component on the basis of the number of times of use of the detachable component.

[9] The plasma irradiation system described in [8], further comprising a second information section which is provided in the base portion constituting body and contains a piece of information which can be read by the control apparatus,
wherein the second information section includes a second memory section for memorizing the number of times of use of the base portion constituting body, and
wherein, in the reading processing, the control apparatus reads the piece of identification information for identifying the detachable component recorded in the identification section and the piece of identification information for identifying the base portion constituting body memorized in the second memory section,
in the memorizing processing, the control apparatus memorizes the number of times of use of the detachable component in the apparatus-side memory section in such a manner that the number of times of use is made to correspond to the read identification information of the detachable component and updates the number of times of use of the base portion constituting body memorized in the second memory section, and
in the permission processing, the control apparatus permits operation of the plasma irradiation device on the basis of the number of times of use of the detachable component memorized in the apparatus-side memory section and the number of times of use of the base portion constituting body memorized in the second memory section.

The above-described plasma irradiation system requires replacement of the detachable component when the number of times of use of the detachable component reaches an upper limit and requires replacement of the base portion constituting body when the number of times of use of the base portion constituting body reaches an upper limit. Therefore, the above-described plasma irradiation system can prompt the user to replace the detachable component on the basis of the number of times of use of the detachable component and can also prompt the user to replace the base portion constituting body on the basis of the number of times of use of the base portion constituting body:

[10] The plasma irradiation system described in any of [6] to [9], wherein, when a predetermined time set beforehand has elapsed in a state in which the control apparatus has still permitted operation of the plasma irradiation device, the control apparatus notifies a user of elapse of the predetermined time.

The above-described plasma irradiation system can notify the user of the fact that the predetermined time has elapsed in a state in which the control apparatus has still permitted operation of the plasma irradiation device.

[11] A plasma irradiation system comprising:
a plasma irradiation device which includes a base portion functioning as a grip portion and a detachable component detachably attached to the base portion and having a discharging opening for discharging plasma;
a control apparatus to which the plasma irradiation device is connected and which controls the plasma irradiation device; and
a dongle which includes a memory section and is connected to the control apparatus,
wherein the control apparatus performs a memorizing processing of updating the number of times of use memorized in the memory section, and a permission processing of permitting operation of the plasma irradiation device on the basis of the number of times of use memorized in the memory section.

The above-described plasma irradiation system can manage the number of times of use of the detachable component by using the dongle.

[12] A control apparatus which is used in a plasma irradiation system including a plasma irradiation device and a package and which controls the plasma irradiation device,
wherein the plasma irradiation device includes a base portion functioning as a grip portion and a detachable component detachably attached to the base portion and having a discharging opening for discharging plasma,
wherein the package comprises:
the detachable component;
a packaging material for packaging the detachable component; and
an information section containing a piece of information which can be read by the control apparatus,
wherein the information section includes a memory section for memorizing the number of times of use of the detachable component and is provided at least on the detachable component, on the packaging material, or in the packaging material, and
wherein the control apparatus performs a reading processing of reading the number of times of use of the detachable component memorized in the memory section, a memorizing processing of updating the number of times of use of the detachable component memorized in the memory section, and a permission processing of permitting operation of the plasma irradiation device on the basis of the number of times of use of the detachable component memorized in the memory section.

The above-described control apparatus can manage the number of times of use of the detachable component.

[13] The control apparatus described in [12],
wherein the plasma irradiation device includes a second information section which is provided in a base portion constituting body constituting the base portion and contains a piece of information which can be read by the control apparatus,
wherein the second information section includes a second memory section for memorizing the number of times of use of the base portion constituting body, and
wherein, in the reading processing, the control apparatus reads the number of times of use of the detachable component memorized in the memory section and the number of times of use of the base portion constituting body memorized in the second memory section,
in the memorizing processing, the control apparatus updates the number of times of use of the detachable component memorized in the memory section and the number of times of use of the base portion constituting body memorized in the second memory section, and
in the permission processing, the control apparatus permits operation of the plasma irradiation device on the basis of the number of times of use of the detachable component memorized in the memory section and the number of times of use of the base portion constituting body memorized in the second memory section.

The above-described control apparatus can manage not only the number of times of use of the detachable component but also the number of times of use of the base portion constituting body.

[14] A control apparatus which is used in a plasma irradiation system including a plasma irradiation device and a package and which controls the plasma irradiation device,
wherein the plasma irradiation device includes a base portion functioning as a grip portion and a detachable component detachably attached to the base portion and having a discharging opening for discharging plasma,
wherein the package comprises:
the detachable component;
a packaging material for packaging the detachable component; and
an information section containing a piece of information which can be read by the control apparatus,
wherein the information section is provided at least on the detachable component, on the packaging material, or in the packaging material and includes an identification section in which a piece of identification information for identifying the detachable component has been recorded, and
wherein the control apparatus performs a reading processing of reading the piece of identification information for identifying the detachable component recorded in the identification section, a memorizing processing of memorizing the number of times of use of the detachable component in an apparatus-side memory section in such a manner that the number of times of use is made to correspond to the read identification information, and a permission processing of permitting operation of the plasma irradiation device on the basis of the number of times of use of the detachable component memorized in the apparatus-side memory section.

The above-described control apparatus can manage the number of times of use of the detachable component.

[15] The control apparatus described in [14],
wherein the plasma irradiation device includes a second information section which is provided in a base portion constituting body constituting the base portion and contains a piece of information which can be read by the control apparatus,
wherein the second information section includes a second memory section for memorizing the number of times of use of the base portion constituting body, and
wherein, in the reading processing, the control apparatus reads the piece of identification information for identifying the detachable component recorded in the identification section and the piece of identification information for identifying the base portion constituting body memorized in the second memory section,
in the memorizing processing, the control apparatus memorizes the number of times of use of the detachable component in the apparatus-side memory section in such a manner that the number of times of use is made to correspond to the read identification information of the detachable component and updates the number of times of use of the base portion constituting body memorized in the second memory section, and
in the permission processing, the control apparatus permits operation of the plasma irradiation device on the basis of the number of times of use of the detachable component memorized in the apparatus-side memory section and the number of times of use of the base portion constituting body memorized in the second memory section.

The above-described control apparatus can manage not only the number of times of use of the detachable component but also the number of times of use of the base portion constituting body.

[16] The control apparatus described in any of to [15], wherein a predetermined time set beforehand has elapsed in a state in which the control apparatus has still permitted operation of the plasma irradiation device, the control apparatus notifies a user of elapse of the predetermined time.

The above-described control apparatus can notify the user of the fact that the predetermined time has elapsed in a state in which the control apparatus has still permitted operation of the plasma irradiation device.

### 1. First Embodiment

### 1-1. Configuration of Plasma Irradiation System 100

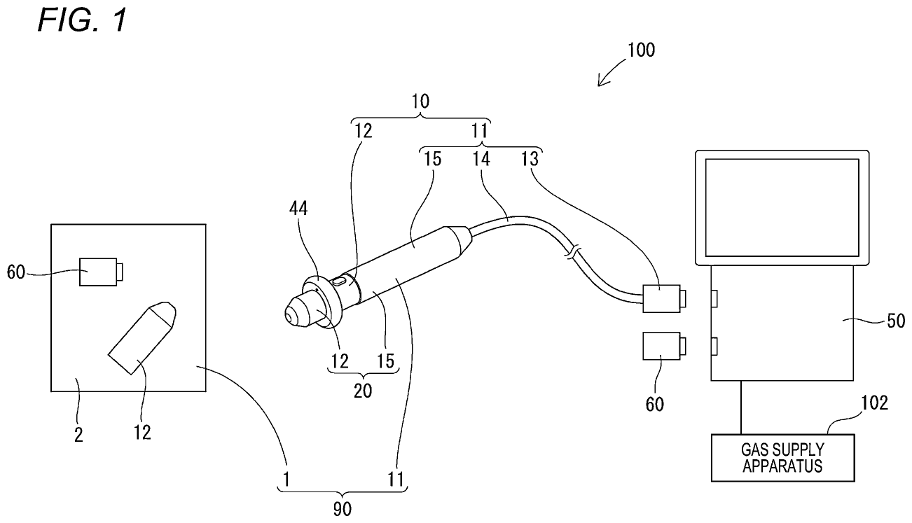

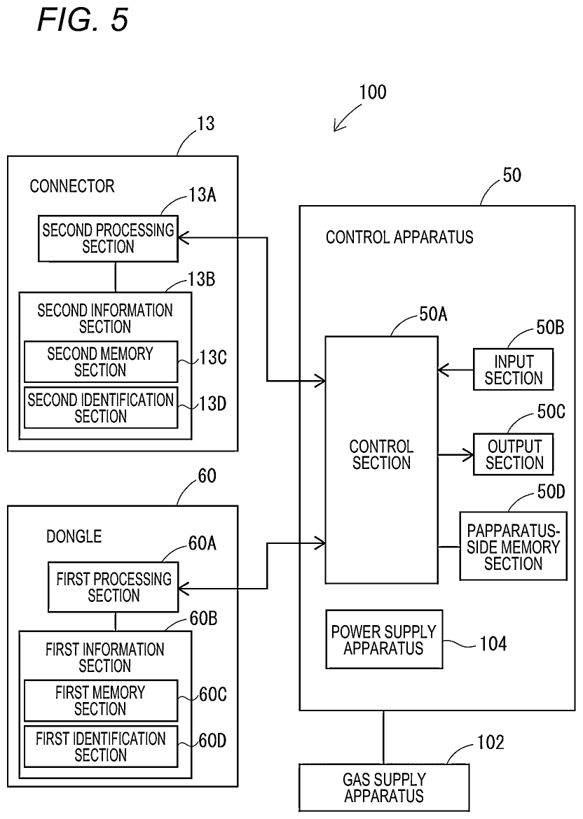

A package 1 used for a plasma irradiation system 100 is disclosed in FIG. 1. The plasma irradiation system 100 is used, for example, in operations such as dental treatment. The plasma irradiation system 100 includes a plasma irradiation device 10 which discharges plasma, a control apparatus 50 which controls the plasma irradiation device 10, a dongle 60 which is connected to the control apparatus 50, a gas supply apparatus 102 which supplies gas to the plasma irradiation device 10, and a power supply apparatus 104 (see FIG. 5) which supplies electric power to the plasma irradiation device 10.

The plasma irradiation device 10 is configured, for example, as a surgical instrument which discharges plasma to a biological tissue to be treated. Upon reception of electric power supplied from the power supply apparatus 104 (see FIG. 5), the plasma irradiation device 10 generates and discharges plasma. The plasma irradiation device 10 discharges low-energy plasma which is low in invasiveness.

The plasma irradiation device 10 includes a base portion constituting body 11 which is connected to the control apparatus 50, and a detachable component 12 which is detachably attached to the base portion constituting body 11.

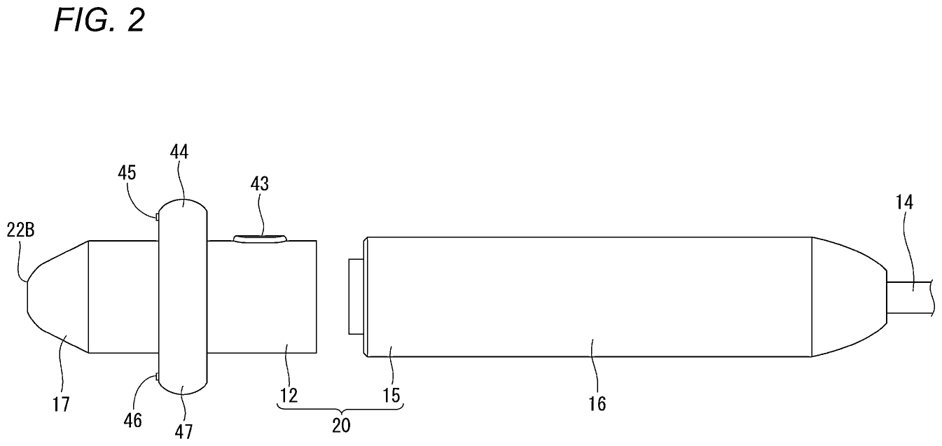

The base portion constituting body 11 includes a connector 13 connected to the control apparatus 50, a cable 14 extending from the connector 13 toward a forward end side, and a base portion 15 provided at a forward end of the cable 14. The cable 14 has flexibility. The cable 14 includes relaying wiring lines, a relaying flow channel, and a sheath which covers the outer circumferences of the relaying wiring lines and the relaying flow channel together. As shown in FIG. 2, the base portion 15 is elongated in a predetermined direction (more specifically, a forward-rearward direction). The base portion 15 functions as a grip portion. The base portion 15 has a first case 16. The first case 16 constitutes the outer shell of the base portion 15. The first case 16 has insulating properties and is formed of, for example, a resin material. The first case 16 has the shape of a tube elongated in the predetermined direction (more specifically, the forward-rearward direction). The detachable component 12 is detachably attached to the base portion 15 (more specifically, to a forward end portion of the base portion 15).

As shown in FIG. 2, the detachable component 12 has a second case 17. The second case 17 constitutes the outer shell of the detachable component 12. The second case 17 has insulating properties and is formed of, for example, a resin material. The second case 17 has a tubular shape. A discharging opening 22B is formed in the detachable component 12. The detachable component 12 and the base portion 15 constitute a plasma irradiation instrument 20.

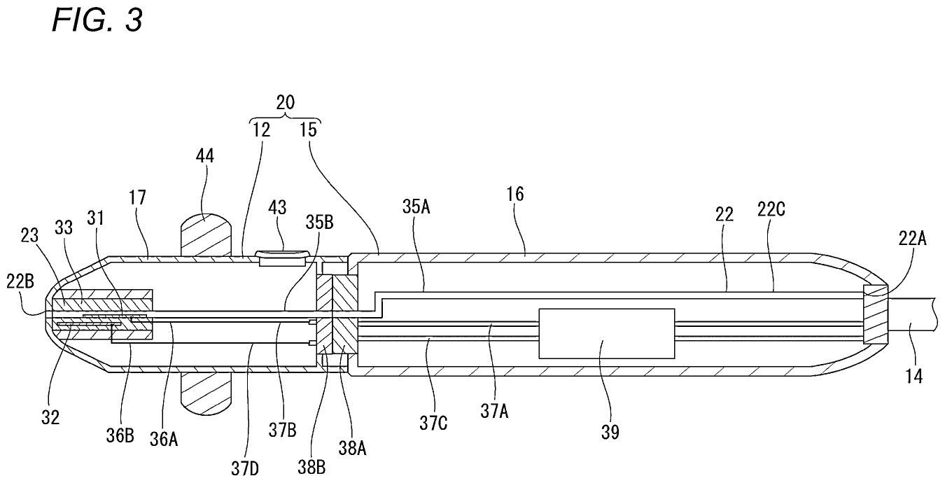

As shown in FIG. 3, the plasma irradiation instrument 20 has a gas leading channel 22 for leading gas toward a forward end portion of the gas leading channel 22, and an electric discharge section 23 for generating plasma within the gas leading channel 22. The gas leading channel 22 leads the gas, which is supplied from the gas supply apparatus 102 via the relaying flow channel of the cable 14, toward the forward end portion of the gas leading channel 22.

As shown in FIG. 3, the gas leading channel 22 has an introduction opening 22A for introducing the gas, a discharging opening 22B for discharging the gas, and a flow channel 22C provided between the introduction opening 22A and the discharging opening 22B. The gas leading channel 22 serves as a guide path into which the gas supplied from the gas supply apparatus 102 is introduced through the introduction opening 22A and which guides the gas, introduced from the introduction opening 22A side, to the discharging opening 22B through the space within the flow channel 22C. The discharging opening 22B is provided at a forward end portion of the plasma irradiation device 10 and is open forward. The gas flowing through the gas leading channel 22 is discharged through the discharging opening 22B. The discharging opening 22B corresponds to the “forward end portion of the gas leading channel 22.”

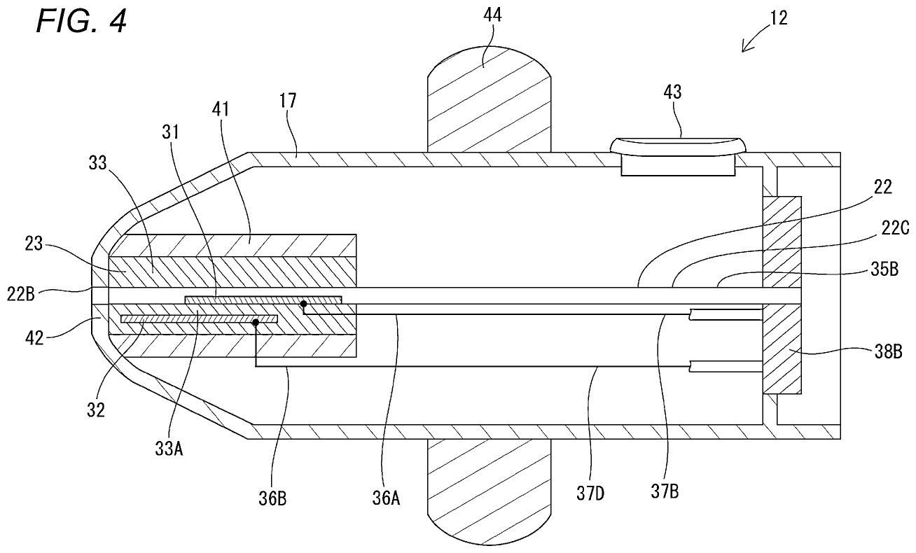

As shown in FIGS. 3 and 4, the electric discharge section 23 is composed of only a first electrode 31, a second electrode 32, and a dielectric member 33 including a dielectric layer 33A present between the first electrode 31 and the second electrode 32. The electric discharge section 23 is hollow and constitutes at least a portion of the gas leading channel 22. The electric discharge section 23 is elongated in the longitudinal direction of the base portion 15 (the forward-rearward direction). The electric discharge section 23 is provided in the detachable component 12. The electric discharge section 23 is disposed on the forward end portion (discharging opening 22B) side in the detachable component 12. Notably, the “forward end portion side in the detachable component 12” means the “forward end portion side in relation to the center of the detachable component 12 in the forward-rearward direction (the longitudinal direction of the base portion 15).”

The first electrode 31 functions as a discharge electrode. The second electrode 32 serves as a ground electrode. Each of the first electrode 31 and the second electrode 32 extends in the longitudinal direction of the base portion 15 (the forward-rearward direction). The first electrode 31 and the second electrode 32 are provided integrally with the dielectric member 33. The first electrode 31 and the second electrode 32 are disposed in such a manner that they are separated from each other and face each other with the dielectric layer 33A of the dielectric member 33 intervening therebetween.

The dielectric member 33 is hollow and constitutes at least a portion of the gas leading channel 22. The dielectric member 33 is elongated in the longitudinal direction of the base portion 15 (the forward-rearward direction). For example, a ceramic material such as alumina, a glass material, a resin material, or the like is preferably used to form the dielectric member 33. A forward end of the dielectric member 33 is located on the forward end side in relation to both the first electrode 31 and the second electrode 32.

Upon application of a voltage between the first electrode 31 and the second electrode 32, the electric discharge section 23 generates plasma within the gas leading channel 22. More specifically, the electric discharge section 23 functions to generate an electric field within the gas leading channel 22 by using a potential difference between the first electrode 31 and the second electrode 32 and generate plasma (more specifically, low-temperature plasma) within the gas leading channel 22 by discharge (more specifically, creeping discharge). In the electric discharge section 23, as a result of the potential of the second electrode 32 being maintained at a fixed reference potential (for example, ground potential of (V) and a periodically changing voltage being applied to the first electrode 31, creeping discharge occurs within the gas leading channel 22, whereby plasma (more specifically, low-temperature plasma) is generated.

The flow channel 22C of the above-described gas leading channel 22 has a first flow channel 35A provided in the base portion 15 and a second flow channel 35B provided in the detachable component 12. The first flow channel 35A is disposed in the first case 16. The first flow channel 35A is disposed on the gas supply apparatus 102 side in relation to the second flow channel 35B. The second flow channel 35B is disposed on the discharging opening 22B side in relation to the first flow channel 35A. The gas supplied from the gas supply apparatus 102 side is introduced to the first flow channel 35A. The first flow channel 35A causes the gas supplied from the gas supply apparatus 102 side to flow to the second flow channel 35B side. The second flow channel 35B leads the gas supplied from the first flow channel 35A side to the discharging opening 22B side and discharges the gas from the discharging opening 22B. Notably, the electric discharge section 23 generates plasma within the second flow channel 35B.

The plasma irradiation instrument 20 has a first wiring line 36A electrically connected to the first electrode 31 and a second wiring line 36B electrically connected to the second electrode 32. The voltage from the power supply apparatus 104 is applied between the first wiring line 36A and the second wiring line 36B. The voltage applied between the first wiring line 36A and the second wiring line 36B is applied between the first electrode 31 and the second electrode 32.

The first wiring line 36A has a first conduction path 37A provided in the base portion 15 and a second conduction path 37B provided in the detachable component 12. The second wiring line 36B has a third conduction path 37C provided in the base portion 15 and a fourth conduction path 37D provided in the detachable component 12. The first conduction path 37A and the third conduction path 37C are disposed in the first case 16. The second conduction path 37B and the fourth conduction path 37D are disposed in the second case 17. Notably, in FIG. 4, specific wiring routes of the second conduction path 37B and the fourth conduction path 37D are omitted.

The base portion 15 has a first connector portion 38A. The first connector portion 38A is provided at a forward end portion of the base portion 15. The detachable component 12 has a second connector portion 38B. The second connector portion 38B is provided at a rear end portion of the detachable component 12. The first connector portion 38A and the second connector portion 38B are connected to each other.

One end of the first flow channel 35A is provided in the first connector portion 38A. The introduction opening 22A is provided at the other end of the first flow channel 35A. One end of the first conduction path 37A and one end of the third conduction path 37C are provided in the first connector portion 38A.

One end of the second flow channel 35B is provided in the second connector portion 38B. The discharging opening 22B is provided at the other end of the second flow channel 35B. One end of the second conduction path 37B and one end of the fourth conduction path 37D are provided in the second connector portion 38B. The other end of the second conduction path 37B is electrically connected to the first electrode 31. The other end of the fourth conduction path 37D is electrically connected to the second electrode 32. The second electrode 32 is connected to the ground via the second wiring line 36B.

When the second connector portion 38B is connected to the first connector portion 38A, supply of gas from the first flow channel 35A to the second flow channel 35B becomes possible, the second conduction path 37B is electrically connected to the first conduction path 37A, and the fourth conduction path 37D is electrically connected to the third conduction path 37C.

For example, a first pipe portion which constitutes the first flow channel 35A and a second pipe portion which constitutes the second flow channel 35B are connected in a state in which gas leakage is prevented. For example, a female terminal is provided on one of the first conduction path 37A and the second conduction path 37B, a male terminal is provided on the other of the first conduction path 37A and the second conduction path 37B, and the female terminal and the male terminal are connected together, whereby the first conduction path 37A and the second conduction path 37B are electrically connected to each other. For example, a female terminal is provided on one of the third conduction path 37C and the fourth conduction path 37D, a male terminal is provided on the other of the third conduction path 37C and the fourth conduction path 37D, and the female terminal and the male terminal are connected together, whereby the third conduction path 37C and the fourth conduction path 37D are electrically connected to each other.

The base portion 15 includes a voltage step-up circuit section 39 which steps up an input voltage and applies the stepped-up voltage to the electric discharge section 23. The voltage step-up circuit section 39 is disposed in the first case 16. The voltage step-up circuit section 39 includes a step-up transformer. The other end of the first conduction path 37A and the other end of the third conduction path 37C are electrically connected to the voltage step-up circuit section 39. The voltage step-up circuit section 39 steps up the input voltage from the power supply apparatus 104 and applies the stepped-up voltage between the first conduction path 37A and the third conduction path 37C. In this manner, the voltage step-up circuit section 39 applies a desired voltage between the first electrode 31 and the second electrode 32.

The voltage step-up circuit section 39 functions as a high frequency voltage generation circuit. The voltage step-up circuit section 39 applies an AC voltage of a predetermined frequency between the first electrode 31 and the second electrode 32 while maintaining the second electrode 32 at a ground potential. The voltage step-up circuit section 39 may employ any of various known circuits so long as the employed circuit can generate a high voltage (for example, a high voltage whose amplitude is 0.5 kV to 10 kV) having a high frequency (for example, about 20 kHz to 300 kHz). Notably, the frequency of the high voltage generated by the voltage step-up circuit section 39 may be fixed to a constant value or may change. Also, the voltage applied between the first electrode 31 and the second electrode 32 by the voltage step-up circuit section 39 may have any waveform so long as the voltage changes periodically. For example, the voltage may be a sinusoidal AC voltage or an AC voltage having a non-sinusoidal waveform (for example, a rectangular waveform, a triangular waveform, or the like).

As shown in FIG. 4, the detachable component 12 has a periphery protection portion 41. The periphery protection portion 41 has insulating properties and is formed of, for example, a resin material. The periphery protection portion 41 covers the outer circumference of the hollow electric discharge section 23. The periphery protection portion 41 has a tubular shape. The periphery protection portion 41 is disposed in the second case 17. The outer circumference of the periphery protection portion 41 is covered by the second case 17.

As shown in FIG. 4, the second case 17 of the detachable component 12 has a forward end protection portion 42. The forward end protection portion 42 has insulating properties and is formed of, for example, a resin material. The forward end protection portion 42 covers a forward end surface of the electric discharge section 23. The forward end protection portion 42 is in contact with the forward end surface of the electric discharge section 23 and fixed to the forward end surface of the electric discharge section 23. The forward end protection portion 42 constitutes a portion of the gas leading channel 22. The discharging opening 22B is formed in a forward end portion of the forward end protection portion 42.

As shown in FIG. 4, the detachable component 12 has a switch 43. The switch 43 is provided on the upper surface side of the second case 17. The switch 43 is an operation portion for switching between a state in which plasma is discharged from the plasma irradiation device 10 (more specifically, from the plasma irradiation instrument 20) and a state in which discharge of plasma is stopped. For example, the switch 43 is a press switch. When the switch 43 is in a pressed state, plasma is discharged, and when the switch 43 is released from the pressed state, discharge of plasma is stopped. Notably, in FIG. 4, the peripheral configuration of the switch 43 (for example, a circuit for controlling the discharge of the electric discharge section 23 on the basis of operation of the switch 43) is omitted. However, general control circuits, etc. may be employed.

As shown in FIG. 1, the plasma irradiation device 10 has a sight 44. The sight 44 is a device which is disposed on the plasma irradiation instrument 20 and is used for taking aim at a plasma irradiation position at the time of plasma irradiation. As shown in FIG. 2, the sight 44 includes light sources 45 and 46 and a main body portion 47. The light sources 45 and 46 are built in the main body portion 47. The main body portion 47 has an annular shape. The main body portion 47 is disposed to surround the outer circumference of the detachable component 12. The main body portion 47 is detachably attached to the plasma irradiation instrument 20. The light sources 45 and 46 emit light having directivity. The sight 44 projects the light from the light sources 45 and 46 toward an object (for example, a biological tissue to be treated) which is irradiated with plasma by the plasma irradiation instrument 20.

As shown in FIG. 5, the dongle 60 is an information medium having a first processing section 60A and a first information section 60B. Each of the first processing section 60A and the first information section 60B includes, for example, an IC (integrated circuit) chip. Namely, the first information section 60B includes a memory circuit for memorizing pieces of information. The first processing section 60A reads pieces of information memorized in the first information section 60B and writes predetermined pieces of information into the first information section 60B. The first processing section 60A can communicate with the control apparatus 50. The first information section 60B corresponds to one example of the “information section.” The first information section 60B contains pieces of information which can be read by the control apparatus 50. The first information section 60B includes a first memory section 60C and a first identification section 60D. The first memory section 60C corresponds to one example of the “memory section.” The first memory section 60C memorizes the number of times of use of the detachable component 12. The piece of identification information for identifying the detachable component 12 which is packaged together with the dongle 60 is memorized in the first identification section 60D beforehand. The model number of the detachable component 12, the production number of the detachable component 12, the serial number of the detachable component 12, the lot number of the detachable component 12, etc. are memorized as the piece of identification information for identifying the detachable component 12. The date and time when the dongle 60 is connected to the control apparatus 50 is memorized in the first information section 60B.

As shown in FIG. 5, the connector 13 includes a second processing section 13A and a second information section 13B. Each of the second processing section 13A and the second information section 13B includes, for example, an IC (integrated circuit) chip. Namely, the second information section 13B includes a memory circuit for memorizing pieces of information. The second processing section 13A reads pieces of information memorized in the second information section 13B and writes predetermined pieces of information into the second information section 13B. The second processing section 13A can communicate with the control apparatus 50. The second information section 13B contains pieces of information which can be read by the control apparatus 50. The second information section 13B includes a second memory section 13C and a second identification section 13D. The second memory section 13C memorizes the number of times of use of the base portion constituting body 11. The piece of identification information for identifying the base portion constituting body 11 is memorized in the second identification section 13D beforehand. The model number of the base portion constituting body 11, the production number of the base portion constituting body 11, the serial number of the base portion constituting body 11, the lot number of the base portion constituting body 11, etc. are memorized as the piece of identification information for identifying the base portion constituting body 11. The date and time when the base portion constituting body 11 is connected to the control apparatus 50 is memorized in the second information section 13B.

As shown in FIG. 5, the control apparatus 50 includes a control section 50A, an input section 50B, an output section 50C, and an apparatus-side memory section 50D.

The control section 50A controls operation of the plasma irradiation device 10. The control section 50A includes, for example, an MCU (micro processor unit). Pieces of reference information for determining whether or not the base portion constituting body 11 and the detachable component 12 are proper parts are memorized in the control section 50A. The pieces of reference information include the model number of the base portion constituting body 11 and the model number of the dongle 60. The control section 50A can communicate with the dongle 60 (more specifically, the first processing section 60A) and the connector 13 (more specifically, the second processing section 13A).

The input section 50B is configured by using, for example, known input devices. Examples of known input devices include a keyboard, a mouse, a touch panel, etc. Pieces of information input to the input section 50B are input from the input section 50B to the control section 50A.

The output section 50C is configured by using, for example, known output devices. Examples of known output devices include a display (for example, a liquid crystal display), a sound output unit (for example, a speaker), etc.

The apparatus-side memory section 50D is constituted by, for example, a known semiconductor memory:

As shown in FIG. 5, the gas supply apparatus 102 is connected to the control apparatus 50. The gas supply apparatus 102 is controlled by the control section 50A of the control apparatus 50. The gas supply apparatus 102 is an apparatus for supplying inert gas such as helium gas (hereinafter also referred to simply as gas). The gas supply apparatus 102 supplies the inert gas to the gas leading channel 22, which will be described later, through, for example, a flexible pipe passage (the relaying flow channel in the cable 14) present between the plasma irradiation instrument 20 and the gas supply apparatus 102. The gas supply apparatus 102 includes a regulator which reduces the pressure of high-pressure gas supplied from, for example, a cylinder or the like and a control section which performs flow rate control.

As shown in FIG. 5, the power supply apparatus 104 is provided in the control apparatus 50. The power supply apparatus 104 is an apparatus for applying a desired voltage between the first electrode 31 and the second electrode 32 of the electric discharge section 23. The power supply apparatus 104 applies an AC voltage having a predetermined frequency between the first electrode 31 and the second electrode 32. Notably, the frequency of the high voltage generated by the power supply apparatus 104 may be fixed to a constant value or may change. Also, the voltage applied between the second electrode 32 and the first electrode 31 by the power supply apparatus 104 may be any voltage so long as it changes periodically, and the voltage may be a sinusoidal AC voltage or an AC voltage having a non-sinusoidal waveform (for example, a rectangular waveform, a triangular waveform, or the like). As shown in FIG. 3, the first electrode 31 is electrically connected to the high-potential-side first wiring line 36A. The second electrode 32 is electrically connected to the low-potential-side second wiring line 36B. The voltage step-up circuit section 39 for voltage step-up is provided in the first wiring line 36A and the second wiring line 36B. The voltage step-up circuit section 39 steps up the voltage input from the power supply apparatus 104 side and applies the stepped-up voltage between the first electrode 31 and the second electrode 32. Notably, the second electrode 32 may or may not be maintained at the ground potential.

The package 1 includes the above-described detachable component 12, the above-described dongle 60, and a packaging material 2. The detachable component 12 and the dongle 60 are packaged together with the packaging material 2. The packaging material 2 is used to package one detachable component 12 and one dongle 60 together. A partition may be provided between the detachable component 12 and the dongle 60. The detachable component 12 and the dongle 60 may be disposed in a common space of the packaging material 2 or may be disposed separately in different spaces of the packaging material 2. The dongle 60 is provided in the packaging material 2. Namely, the first information section 60B of the dongle 60 is provided in the packaging material 2. Since the detachable component 12 and the dongle 60 are packaged together by the packaging material 2 as described above, the first information section 60B of the dongle 60 is made to correspond to the detachable component 12.

The package 1 constitutes the plasma irradiation module 90 together with the base portion constituting body 11.

### 1-2. Operation of Plasma Irradiation System 100

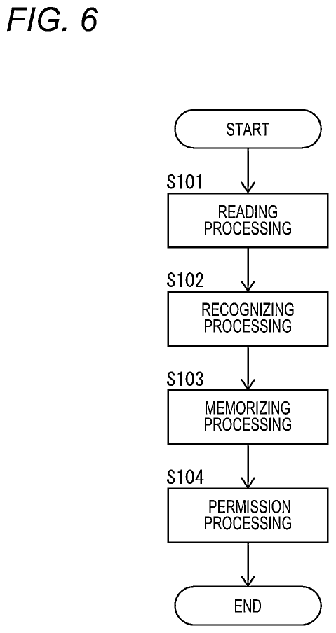

The control section 50A of the control apparatus 50 performs the processing shown in FIG. 6 when a start condition is satisfied. The start condition may be, for example, that the power supply of the control apparatus 50 has been switched to an ON state or that a start operation has been performed.

The control section 50A performs a reading processing in step S101. The reading processing is a processing for reading the piece of identification information for identifying the base portion constituting body 11 and the piece of identification information for identifying the detachable component 12. The control section 50A reads the piece of identification information for identifying the detachable component 12 from the dongle 60 connected to the control apparatus 50. After having read the piece of identification information for identifying the base portion constituting body 11 and the piece of identification information for identifying the detachable component 12, the control section 50A proceeds to a recognizing processing of step S102.

The recognizing processing is a processing for recognizing the base portion constituting body 11 and the dongle 60. The recognizing processing includes a base portion constituting body recognizing processing for recognizing the base portion constituting body 11 and a dongle recognizing processing for recognizing the dongle 60. In the present embodiment, the dongle recognizing processing is performed after recognition of the base portion constituting body 11 by the base portion constituting body recognizing processing. Notably, the base portion constituting body recognizing processing may be performed after recognition of the dongle 60 by the dongle recognizing processing. Also, the base portion constituting body recognizing processing and the dongle recognizing processing may be performed in parallel.

The control section 50A performs the following operation in the base portion constituting body recognizing processing. The control section 50A determines whether or not the base portion constituting body 11 connected to the control apparatus 50 is a proper part on the basis of the reference information and the model number of the base portion constituting body 11 read by the reading processing. In the case where the read model number of the base portion constituting body 11 is contained in the model numbers of the base portion constituting body 11 in the reference information, the control section 50A determines that the base portion constituting body 11 is a proper part. In the case where the read model number of the base portion constituting body 11 is not contained in the model numbers of the base portion constituting body 11 in the reference information, the control section 50A determines that the base portion constituting body 11 is not a proper part. In the case where the control section 50A determines that the base portion constituting body 11 is not a proper part, the control section 50A does not permit operation of the plasma irradiation device 10. In the case where the control section 50A determines that the base portion constituting body 11 is a proper part (namely, the control section 50A recognizes the base portion constituting body 11), the control section 50A proceeds to a dongle recognizing processing.

The control section 50A performs the following operation in the dongle recognizing processing. The control section 50A determines whether or not the detachable component 12 made to correspond to the dongle 60 connected to the control apparatus 50 is a proper part on the basis of the reference information and the model number of the detachable component 12 read by the reading processing. In the case where the read model number of the detachable component 12 is contained in the model numbers of the detachable component 12 in the reference information, the control section 50A determines that the detachable component 12 is a proper part. In the case where the read model number of the detachable component 12 is not contained in the model numbers of the detachable component 12 in the reference information, the control section 50A determines that the detachable component 12 is not a proper part. In the case where the control section 50A determines that the dongle 60 is not a proper part, the control section 50A does not permit operation of the plasma irradiation device 10. In the case where the control section 50A determines that the dongle 60 is a proper part (namely, the control section 50A recognizes the dongle 60), the control section 50A proceeds to a memorizing processing in step S103.

The control section 50A performs the following operation in the memorizing processing. The control section 50A updates the number of times of use of the detachable component 12 memorized in the first memory section 60C of the recognized dongle 60. More specifically, the control section 50A adds one to the number of times of use of the detachable component 12 memorized in the first memory section 60C of the dongle 60. Notably, the initial value of the number of times of use of the detachable component 12 is 0. Also, the control section 50A updates the number of times of use of the base portion constituting body 11 memorized in the second memory section 13C of the recognized base portion constituting body 11. More specifically, the control section 50A adds one to the number of times of use of the base portion constituting body 11 memorized in the second memory section 13C of the base portion constituting body 11. Notably, the initial value of the number of times of use of the base portion constituting body 11 is 0. After completion of the memorizing processing, the control section 50A proceeds to a permission processing of step S104.

The control section 50A performs the following operation in the permission processing. In the case where the dongle 60 and the base portion constituting body 11 are recognized, the control section 50A permits operation of the plasma irradiation device 10 on the basis of the number of times of use of the detachable component 12 memorized in the first memory section 60C and the number of times of use of the base portion constituting body 11 memorized in the second memory section 13C.

In the case where the control section 50A determines that the number of times of use of the detachable component 12 is equal to or greater than a first upper limit number of times (for example, 4 times), the control section 50A does not permit operation of the plasma irradiation device 10. The first upper limit number of times is a value set beforehand and may be a fixed value or an arbitrarily set value.

In the case where the control section 50A determines that the number of times of use of the base portion constituting body 11 is equal to or greater than a second upper limit number of times (for example, 100 times), the control section 50A does not permit operation of the plasma irradiation device 10. The second upper limit number of times is a value set beforehand and may be a fixed value or an arbitrarily set value.

The control section 50A permits operation of the plasma irradiation device 10 in the case where the number of times of use of the detachable component 12 is less than the first upper limit number of times and the number of times of use of the base portion constituting body 11 is less than the second upper limit number of times. More specifically, the control section 50A permits operation of the plasma irradiation device 10 after performing a predetermined operation test. A permitted state in which operation of the plasma irradiation device 10 is permitted is a state in which the plasma irradiation device discharges plasma in response to a predetermined irradiation operation (in the present embodiment, an operation of pressing the switch 43). In the permitted state, in response to the predetermined irradiation operation, the gas supplied from the gas supply apparatus 102 flows within the gas leading channel 22 toward the discharging opening 22B. Also, in the permitted state, in response to the predetermined irradiation operation, the voltage step-up circuit section 39 applies a voltage between the first electrode 31 and the second electrode 32 by using the electric power from the power supply apparatus 104. As a result, the electric discharge section 23 generates plasma within the gas leading channel 22. The plasma generated within the gas leading channel 22 is discharged from the discharging opening 22B.

Notably, the control section 50A may be configured such that, even when the number of times of use of the detachable component 12 is less than the first upper limit number of times (for example, 4 times), if the number of times of use of the detachable component 12 is equal to or greater than a first warning number of times (for example, 2 times), the control section 50A notifies a user of the fact that the number of times of use of the detachable component 12 is equal to or greater than the first warning number of times by using the output section 50C or the control section 50A permits operation of the plasma irradiation device 10 on the condition that a first confirmation operation has been performed. The first warning number of times may be a fixed value or an arbitrarily set value.

Also, the control section 50A may be configured such that, even when the number of times of use of the base portion constituting body 11 is less than the second upper limit number of times (for example, 100 times), if the number of times of use of the base portion constituting body 11 is equal to or greater than a second warning number of times (for example, 90 times), the control section 50A notifies the user of the fact that the number of times of use of the base portion constituting body 11 is equal to or greater than the second warning number of times by using the output section 50C or the control section 50A permits operation of the plasma irradiation device 10 on the condition that a second confirmation operation has been performed. The second warning number of times may be a fixed value or an arbitrarily set value.

The control section 50A ends the permitted state when an end condition is satisfied. The end condition may be that the power supply of the control apparatus 50 has been switched to an OFF state, that an end operation has been performed, or that the base portion constituting body 11 has been disconnected from the control apparatus 50.

The control section 50A measures a time which elapses after having established the permitted state. When a warning time has elapsed after establishment of the permitted state, the control section 50A notifies the user of the fact that the warning time has elapsed by using the output section 50C. The warning time may be a time which is fixedly determined or a time which can be set arbitrarily. The warning time is, for example, 24 hours.

The control section 50A may measure a time which elapses after having established the permitted state and add one to the number of times of use of the detachable component 12, which is memorized in the first memory section 60C of the dongle 60, when a limit time has elapsed after establishment of the permitted state. The limit time may be the same as or different from the warning time. The limit time may be a time which is fixedly determined or a time which can be set arbitrarily. The limit time is, for example, 24 hours.

Notably, the control section 50A may be configured such that, when the control section 50A determines that the base portion constituting body 11 is not connected to the control apparatus 50, the control section 50A notifies, by using the output section 50C, the user of the fact that the base portion constituting body 11 is not connected to the control apparatus 50. Also, the control section 50A may be configured such that, when the control section 50A determines that the dongle 60 is not connected to the control apparatus 50, the control section 50A notifies, by using the output section 50C, the user of the fact that the dongle 60 is not connected to the control apparatus 50.

### 1-3. Examples of Effects

The dongle 60 including the first memory section 60C is provided in the packaging material 2, with which the detachable component 12 is packaged, whereby the dongle 60 is made to correspond to the detachable component 12. Namely, this configuration enables management of the number of times of use of the detachable component 12 by the first memory section 60C made to correspond to the detachable component 12. Thus, the package 1 facilitates management of the number of times of use of the detachable component 12.

Furthermore, the electric discharge section 23 is provided in the detachable component 12. In this configuration, since the electric discharge section 23 is disposed near the discharging opening 22B, plasma is more likely to be generated in a region near the discharging opening 22B.

Furthermore, the first information section 60B includes a memory circuit for memorizing pieces of information and is provided in the packaging material 2. The pieces of information memorized in the memory circuit of the first information section 60B is likely to receive an influence of electromagnetic noise produced stemming from the voltage applied to the electric discharge section 23. Since the package 1 is configured such that the first information section 60B is provided in the packaging material 2, the first information section 60B can be separated from the detachable component in which the electric discharge section 23 is provided. Therefore, the package 1 can reduce the influence on the information in the first information section 60B of the electromagnetic noise produced stemming from the voltage applied to the electric discharge section 23.

The plasma irradiation module 90 can manage the number of times of use of the base portion constituting body 11 by using the second memory section 13C. Namely, the plasma irradiation module 90 can facilitate management of not only the number of times of use of the detachable component 12 but also the number of times of use of the base portion constituting body 11.

Since the plasma irradiation system 100 permits operation of the plasma irradiation device 10 on the basis of the number of times of use of the detachable component 12, upon arrival of a time at which operation of the plasma irradiation device 10 is not permitted, a new dongle 60 becomes necessary. For preparation of a new dongle 60, a new package 1 must be opened. Opening of the new package 1 results in replacement of the detachable component 12. Namely, the plasma irradiation system 100 can prompt the user to replace the detachable component 12 on the basis of the number of times of use of the detachable component 12.

Furthermore, in the plasma irradiation system 100, when the number of times of use of the detachable component 12 reaches the first upper limit number of times, replacement of the detachable component 12 becomes necessary and, when the number of times of use of the base portion constituting body 11 reaches the second upper limit number of times, replacement of the base portion constituting body 11 becomes necessary. Therefore, the plasma irradiation system 100 can prompt the user to replace the detachable component 12 on the basis of the number of times of use of the detachable component 12 and can also prompt the user to replace the base portion constituting body 11 on the basis of the number of times of use of the base portion constituting body 11.

Furthermore, the plasma irradiation system 100 can notify the user of the fact that the predetermined time has elapsed in a state in which operation of the plasma irradiation device 10 has been still permitted.

### 2. Second Embodiment

In the first embodiment, the plasma irradiation system is configured such that the information sections are read by the control apparatus through wired communication. However, the plasma irradiation system may be configured such that the information sections are read by the control apparatus through wireless communication. In the second embodiment, an example in which the information sections are read by the control apparatus through wireless communication will be described. Notably, in the description of the second embodiment, configurational elements which are identical to those of the first embodiment are denoted by the same reference signs and their detailed descriptions are omitted.

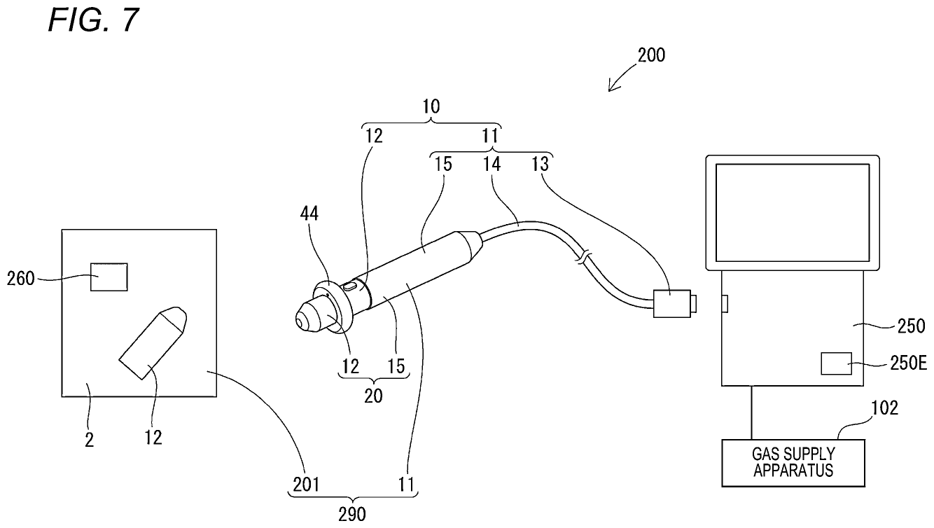

As shown in FIG. 7, a plasma irradiation system 200 of a second embodiment includes the plasma irradiation device 10, a control apparatus 250, and a package 201. The package 201 constitutes a plasma irradiation module 290 together with the base portion constituting body 11.

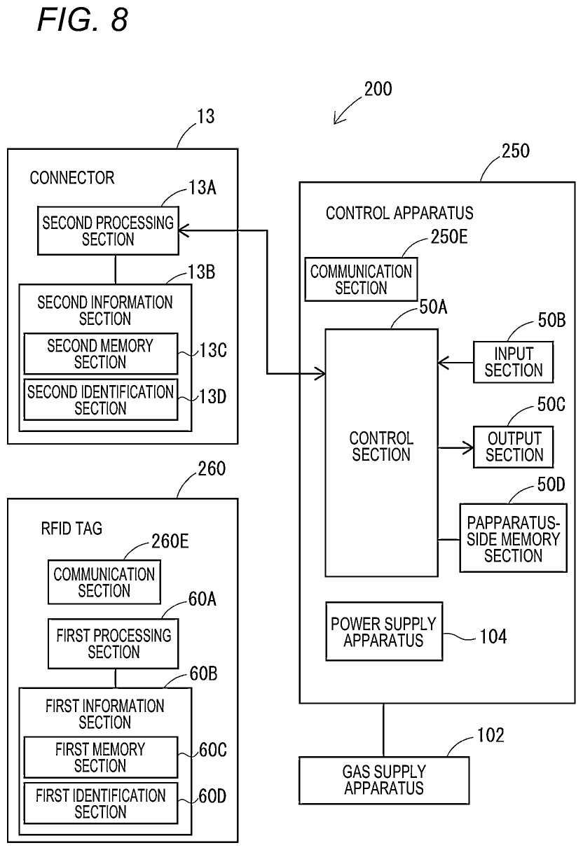

As shown in FIG. 7, the package 201 includes the detachable component 12, the packaging material 2, and an RFID tag 260. As shown in FIG. 8, the RFID tag 260 includes the first processing section 60A and the first information section 60B, which have been described in the first embodiment. Furthermore, the RFID tag 260 includes a communication section 260E. The communication section 260E is, for example, an antenna.

As shown in FIGS. 7 and 8, the control apparatus 250 differs from the control apparatus 50 of the first embodiment mainly in the point that the control apparatus 250 includes a communication section 250E. The communication section 250E is a reader writer which performs wireless communication (more specifically, short-distance wireless communication) with the RFID tag 260. The communication section 250E has a function of reading pieces of information from the RFID tag 260 and a function of writing pieces of information into the RFID tag 260.

Operation of the plasma irradiation system 200 is the same as the operation of the plasma irradiation system 100 described in the first embodiment.

As described above, the information medium (in the present embodiment, the RFID tag) including the first information section 60B may be configured to perform wireless communication with the control apparatus 250. The configuration allows the control apparatus 250 to read pieces of information from the first information section 60B without necessitating wired connection of the information medium to the control apparatus 250.

### 3. Third Embodiment

In a third embodiment, a configuration for managing the number of times of use of the detachable component 12 on the basis of the piece of identification information for identifying the detachable component 12 will be described. Notably, in the description of the third embodiment, configurational elements which are identical to those of the first embodiment are denoted by the same reference signs and their detailed descriptions are omitted.

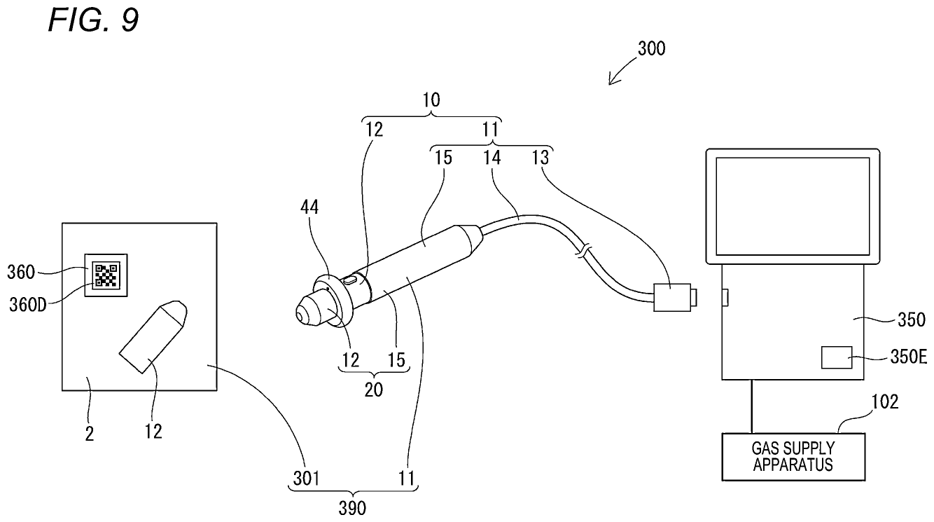

As shown in FIG. 9, a plasma irradiation system 300 of a third embodiment includes the plasma irradiation device 10, a control apparatus 350, and a package 301. The package 301 constitutes a plasma irradiation module 390 together with the base portion constituting body 11.

As shown in FIG. 9, the package 301 includes the detachable component 12, the packaging material 2, and an information medium 360. The information medium 360 includes a first identification section 360D on which the piece of identification information for identifying the detachable component 12 has been recorded. The information medium 360 is formed of a sheet material on which the identification information is printed. The identification information is, for example, a two-dimensional code such as QR code (registered trademark). The information medium 360 is provided on the packaging material 2. The information medium 360 is fixed to the packaging material 2. The information medium 360 is bonded to the surface of the packaging material 2 by means of adhesive.

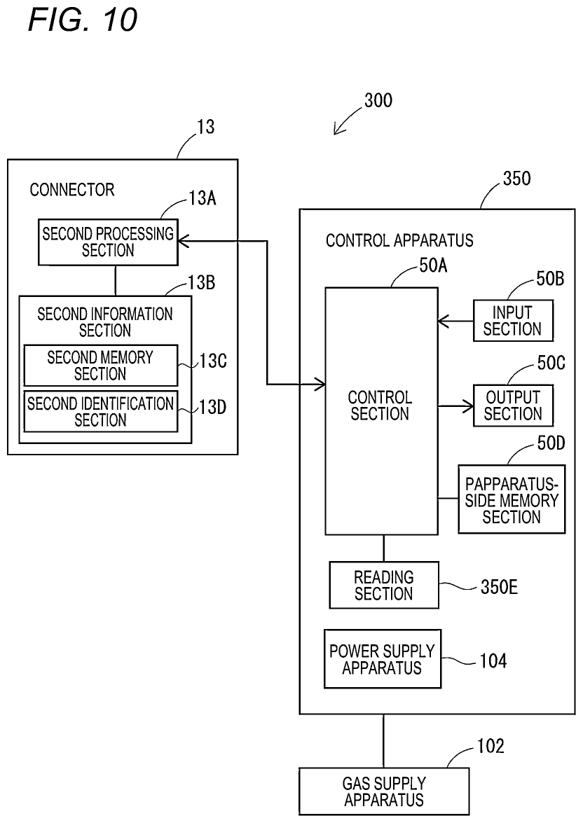

As shown in FIGS. 9 and 10, the control apparatus 350 differs from the control apparatus 50 of the first embodiment mainly in the point that the control apparatus 350 includes a reading section 350E. The reading section 350E is, for example, a reader for reading the two-dimensional code. The reading section 350E reads a piece of information provided on the information medium 360.

Operation of the plasma irradiation system 300 is as follows. In the reading processing, the control apparatus 350 reads the piece of identification information for identifying the detachable component 12 recorded in the first identification section 360D by using the reading section 350E. Also, the control apparatus 350 reads the piece of identification information for identifying the base portion constituting body 11 connected to the control apparatus 350.

The recognizing processing is the same as the recognizing processing described in the first embodiment.

In the memorizing processing, the control apparatus 350 memorizes the number of times of use of the detachable component 12 in the apparatus-side memory section 50D in such a manner that the number of times of use is made to correspond to the read identification information of the detachable component 12. For example, in the case where the read identification information of the detachable component 12 is the identification information read for the first time, the control apparatus 350 memorizes “1” as the number of times of use of the detachable component 12 in such a manner that it is made to correspond to that identification information. In the case where the read identification information of the detachable component 12 is the same as the identification information read formerly, the control apparatus 350 updates the number of times of use of the detachable component 12 corresponding to that identification information. Specifically, the control apparatus 350 adds “1” to the number of times of use of the detachable component 12 corresponding to that identification information.

The determination as to whether or not the read identification information is the identification information read for the first time is made on the basis of, for example, the serial number of the detachable component 12. The determination as to whether or not the read identification information is the identification information read for the first time may be made by using the serial number of the detachable component 12 only or in combination with at least one of the model number of the detachable component 12, the production number of the detachable component 12, and the lot number of the detachable component 12.

In the permission processing, the control apparatus 350 permits operation of the plasma irradiation device 10 on the basis of the number of times of use of the detachable component 12 memorized in the apparatus-side memory section 50D and the number of times of use of the base portion constituting body 11 memorized in the second memory section 13C.

As described above the information medium 360 including the first identification section 360D is bonded to the surface of the packaging material 2, with which the detachable component 12 is packaged, whereby the first identification section 360D is made to correspond to that detachable component 12. Namely, this configuration makes it possible to specify the piece of identification information for identifying the detachable component 12 on the control apparatus 350 side by using the first identification section 360D made to correspond to the detachable component 12 and to manage the number of times of use of the detachable component 12 for each piece of identification information. As described above, the package 1 can facilitate management of the number of times of use of the detachable component 12.

Since the plasma irradiation system 300 permits operation of the plasma irradiation device 10 on the basis of the number of times of use of the detachable component 12, upon arrival of a time at which operation of the plasma irradiation device 10 is not permitted, a new information medium 360 becomes necessary. For preparation of a new information medium 360, a new package 301 must be opened. Opening of the new package 301 results in replacement of the detachable component 12. Namely, the plasma irradiation system 300 can prompt the user to replace the detachable component 12 on the basis of the number of times of use of the detachable component 12.

Furthermore, in the plasma irradiation system 300, when the number of times of use of the detachable component 12 reaches the first upper limit number of times, replacement of the detachable component 12 becomes necessary and, when the number of times of use of the base portion constituting body 11 reaches the second upper limit number of times, replacement of the base portion constituting body 11 becomes necessary. Therefore, the plasma irradiation system 300 can prompt the user to replace the detachable component 12 on the basis of the number of times of use of the detachable component 12 and can also prompt the user to replace the base portion constituting body 11 on the basis of the number of times of use of the base portion constituting body 11.

### 4. Fourth Embodiment

In the third embodiment, the identification section is provided on the packaging material. However, the identification section may be provided on the detachable component 12. In a fourth embodiment, a configuration in which the identification section is provided on the detachable component will be described. Notably, in the description of the fourth embodiment, configurational elements which are identical to those of the third embodiment are denoted by the same reference signs and their detailed descriptions are omitted.

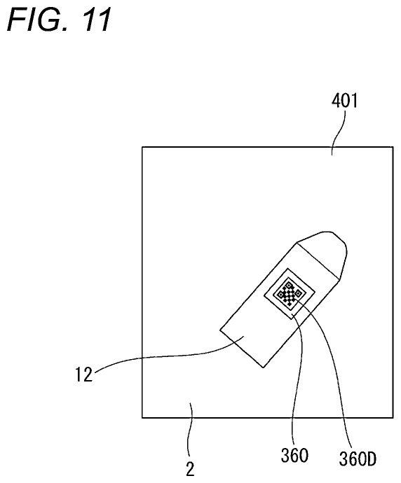

As shown in FIG. 11, a package 401 of the fourth embodiment includes the detachable component 12, the packaging material 2, and the information medium 360. The information medium 360 is provided on the detachable component 12. The information medium 360 is fixed to the detachable component 12. The information medium 360 is bonded to the surface of the detachable component 12 by means of adhesive.

In the above-described configuration as well, the first identification section 360D of the information medium 360 can be made to correspond to the detachable component 12. Therefore, this configuration makes it possible to specify the piece of identification information for identifying the detachable component 12 on the control apparatus 350 side by using the first identification section 360D and to manage the number of times of use of the detachable component 12 for each piece of identification information. As described above, the package 401 can facilitate management of the number of times of use of the detachable component 12.

### 5. Fifth Embodiment

In a fifth embodiment, an example in which a forward end surface of the electric discharge section 23 is exposed will be described. Notably, in the description of the second embodiment, configurational elements which are identical to those of the first embodiment are denoted by the same reference signs and their detailed descriptions are omitted.

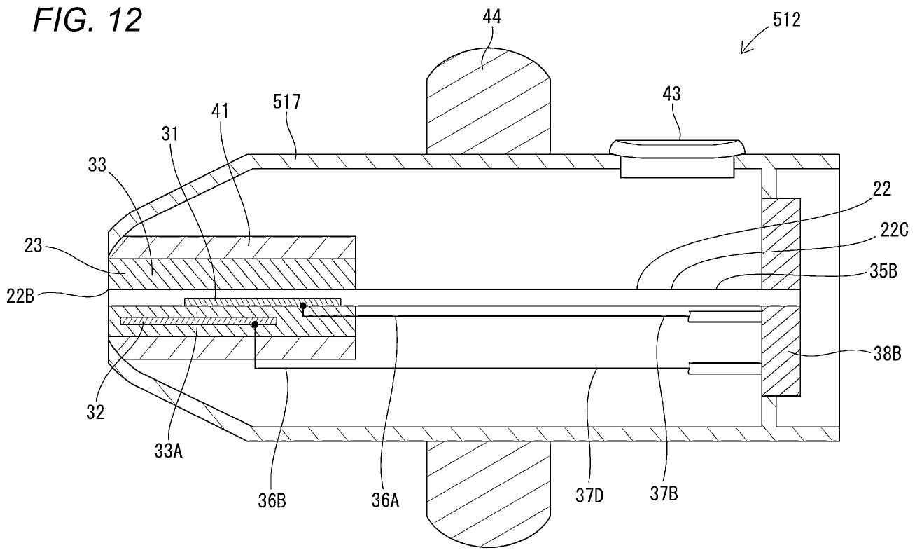

A detachable component 512 of the fifth embodiment shown in FIG. 12 has a structure obtained by removing the forward end protection portion 42 from the detachable component 12 of the first embodiment. A second case 517 of the detachable component 512 constitutes the outer shell of the detachable component 512. The second case 517 has a tubular shape. The second case 517 has a structure obtained by removing the forward end protection portion 42 from the second case 17 of the first embodiment.

The outer circumference of the electric discharge section 23 is covered by the periphery protection portion 41. The outer circumference of the periphery protection portion 41 is covered by the second case 517.

A forward end of the dielectric member 33 in the electric discharge section 23 is exposed at a forward end of the detachable component 512. Also, the forward end of the dielectric member 33 is disposed on the forward end portion side in relation to both the first electrode 31 and the second electrode 32. This configuration makes it possible to secure insulating properties on the forward end side of the first electrode 31 and the second electrode 32 by the dielectric member 33, while enabling the electric discharge section 23 to be disposed on the forward end portion side in the detachable component 12.

### 6. Sixth Embodiment

In a sixth embodiment, an example in which a protection member is attached to the forward end surface of the electric discharge section 23 described in the fifth embodiment will be described. Notably, in the description of the sixth embodiment, configurational elements which are identical to those of the fifth embodiment are denoted by the same reference signs and their detailed descriptions are omitted.

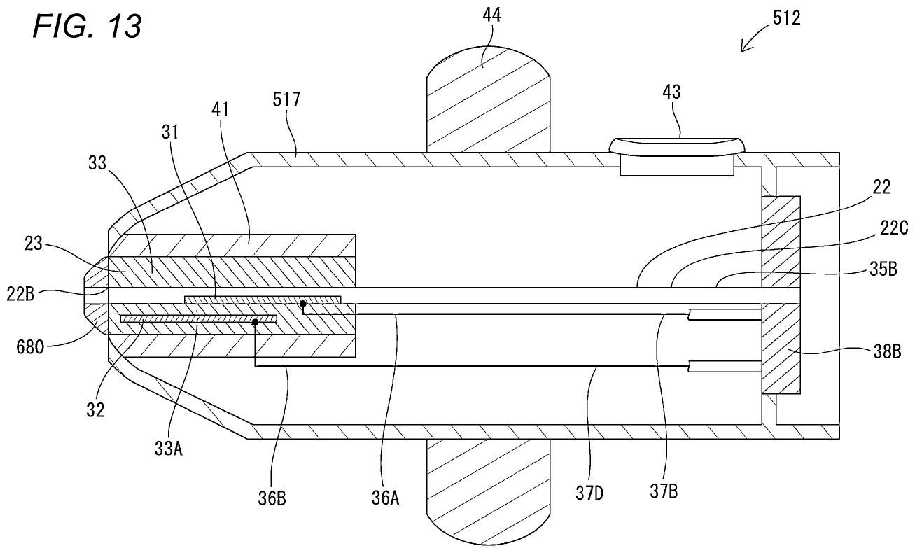

A plasma irradiation device of the sixth embodiment is the same as the plasma irradiation device of the fifth embodiment except the point that the plasma irradiation device of the sixth embodiment has a protection member 680 (see FIG. 13). As shown in FIG. 13, the protection member 680 is attached to the detachable component 512 described in the fifth embodiment. The protection member 680 has an annular shape. The protection member 680 is attached to the forward end surface of the electric discharge section 23 (more specifically, the forward end surface of the dielectric member 33) in such a manner that the protection member 680 does not close the discharging opening 22B. The protection member 680 covers the forward end surface of the electric discharge section 23 (more specifically, the forward end surface of the dielectric member 33). This configuration makes it possible to secure insulating properties on the forward end side of the electric discharge section 23, while protecting the forward end surface of the electric discharge section 23 by the protection member 680.

### Other Embodiments

The present invention is not limited to the embodiments described by the above description with reference to the drawings, and, for example, embodiments which will be described below are contained in the technical scope of the present invention. Also, various features of the above-described embodiments and the embodiments which will be described below may be combined in any manner so long as a conflict does not occur.

In the above-described embodiments, the control apparatus is configured to perform the permission processing after performing the memorizing processing. However, the control apparatus may be configured to perform the memorizing processing after performing the permission processing. Namely, the control apparatus may permit operation of the plasma irradiation device on the basis of the number of times of use before update and then update the number of times of use after perming the operation.

In the above-described embodiments, the detachable component is configured to have the electric discharge section. However, the detachable component may be configured such that the electric discharge section is not provided therein. The detachable component may be, for example, a cap.

In the above-described first embodiment, the dongle is configured to be connected directly to the control apparatus. However, the dongle may be configured to be connectable to the base portion constituting body and be connected to the control apparatus via the base portion constituting body.

In the above-described fourth embodiment, the configuration in which the information medium including the identification section is provided on the detachable component is employed as the configuration in which the identification section is provided on the detachable component. However, other configurations may be employed. For example, the identification section may be printed directly on the detachable component.

Notably, the embodiments disclosed this time should be considered to be illustrative and not to be restrictive in all aspects. The scope of the present invention is not limited to the embodiments disclosed this time, and it is intended that the present invention encompasses all modifications within the range shown by the claims and the range of equivalents of the claims.

### REFERENCE SIGNS LIST

1: package
2: packaging material
10: plasma irradiation device
11: base portion constituting body
12: detachable component
13B: second information section
13C: second memory section
13D: second identification section
15: base portion
22B: discharging opening
23: electric discharge section
39: voltage step-up circuit section
50: control apparatus
50D: apparatus-side memory section
60: dongle
60B: first information section (information section)
60C: first memory section (memory section)
60D: first identification section (identification section)
90: plasma irradiation module
100: plasma irradiation system
200: plasma irradiation system
201: package
250: control apparatus
250E: communication section
260: RFID tag
260E: communication section
290: plasma irradiation module
300: plasma irradiation system
301: package
350: control apparatus
360: information medium
360D: first identification section (identification section)
390: plasma irradiation module
401: package
512: detachable component

1. A package used for a plasma irradiation system,
wherein the plasma irradiation system comprises:
a plasma irradiation device which includes a base portion functioning as a grip portion, and a detachable component which is detachably attached to the base portion and has a discharging opening for discharging plasma; and
a control apparatus to which the plasma irradiation device is connected and which controls the plasma irradiation device,
wherein the package comprises:
the detachable component;
a packaging material for packaging the detachable component; and
an information section containing a piece of information readable by the control apparatus, and
wherein the information section is provided at least on the detachable component, on the packaging material, or in the packaging material and includes at least one of a memory section for memorizing the number of times of use of the detachable component, and an identification section in which a piece of identification information for identifying the detachable component has been recorded.

2. A package according to claim 1, wherein the plasma irradiation device has an electric discharge section for generating plasma, and the electric discharge section is provided in the detachable component.

3. A package according to claim 2, wherein the information section includes a memory circuit for memorizing a piece of information and is provided at least on the packaging material or in the packaging material.

4. A plasma irradiation module comprising:
a package according to claim 1; and
a base portion constituting body which constitutes the base portion.

5. A plasma irradiation module according to claim 4, further comprising a second information section which is provided in the base portion constituting body and contains a piece of information readable by the control apparatus, wherein the second information section includes at least one of a second memory section for memorizing the number of times of use of the base portion constituting body, and a second identification section in which a piece of identification information for identifying the base portion constituting body has been recorded.

6. A plasma irradiation system comprising:
a plasma irradiation module according to claim 4; and
the control apparatus,
wherein the information section includes the memory section, and
wherein the control apparatus performs a reading processing of reading the number of times of use of the detachable component memorized in the memory section, a memorizing processing of updating the number of times of use of the detachable component memorized in the memory section, and a permission processing of permitting operation of the plasma irradiation device on the basis of the number of times of use of the detachable component memorized in the memory section.

7. A plasma irradiation system according to claim 6, further comprising a second information section which is provided in the base portion constituting body and contains a piece of information readable by the control apparatus,
wherein the second information section includes a second memory section for memorizing the number of times of use of the base portion constituting body, and
wherein, in the reading processing, the control apparatus reads the number of times of use of the detachable component memorized in the memory section and the number of times of use of the base portion constituting body memorized in the second memory section,
in the memorizing processing, the control apparatus updates the number of times of use of the detachable component memorized in the memory section and the number of times of use of the base portion constituting body memorized in the second memory section, and
in the permission processing, the control apparatus permits operation of the plasma irradiation device on the basis of the number of times of use of the detachable component memorized in the memory section and the number of times of use of the base portion constituting body memorized in the second memory section.

8. A plasma irradiation system comprising:
a plasma irradiation module according to claim 4; and
the control apparatus,
wherein the information section includes the identification section, and
wherein the control apparatus performs a reading processing of reading the piece of identification information for identifying the detachable component recorded in the identification section, a memorizing processing of memorizing the number of times of use of the detachable component in an apparatus-side memory section in such a manner that the number of times of use is made to correspond to the read identification information, and a permission processing of permitting operation of the plasma irradiation device on the basis of the number of times of use of the detachable component memorized in the apparatus-side memory section.

9. A plasma irradiation system according to claim 8, further comprising a second information section which is provided in the base portion constituting body and contains a piece of information readable by the control apparatus,
wherein the second information section includes a second memory section for memorizing the number of times of use of the base portion constituting body, and
wherein, in the reading processing, the control apparatus reads the piece of identification information for identifying the detachable component recorded in the identification section and the number of times of use of the base portion constituting body memorized in the second memory section,
in the memorizing processing, the control apparatus memorizes the number of times of use of the detachable component in the apparatus-side memory section in such a manner that the number of times of use is made to correspond to the read identification information of the detachable component and updates the number of times of use of the base portion constituting body memorized in the second memory section, and
in the permission processing, the control apparatus permits operation of the plasma irradiation device on the basis of the number of times of use of the detachable component memorized in the apparatus-side memory section and the number of times of use of the base portion constituting body memorized in the second memory section.

10. A plasma irradiation system according to a claim 6, wherein, when a predetermined time set beforehand has elapsed in a state in which the control apparatus has still permitted operation of the plasma irradiation device, the control apparatus notifies a user of elapse of the predetermined time.

11. A plasma irradiation system comprising:
a plasma irradiation device which includes a base portion functioning as a grip portion and a detachable component detachably attached to the base portion and having a discharging opening for discharging plasma;
a control apparatus to which the plasma irradiation device is connected and which controls the plasma irradiation device; and
a dongle which includes a memory section and is connected to the control apparatus,
wherein the control apparatus performs a memorizing processing of updating the number of times of use memorized in the memory section, and a permission processing of permitting operation of the plasma irradiation device on the basis of the number of times of use memorized in the memory section.
